(19) United States
(12) Patent Application Publication
(10) Pub. No.: US 2020/0345614 A1
(43) Pub. Date: Nov. 5, 2020

(54) BODYPAINTING INKS, BODYPAINTING INSTRUMENTS AND METHODS FOR MANUFACTURING BODYPAINTING INKS

(71) Applicants: Kotobuki & Co., Ltd., Kawagoe-shi (JP); Hobbic Study Incorporated, Osaka (JP)

(72) Inventor: Yuichi Takeuchi, Osaka (JP)

(21) Appl. No.: 16/399,772

(22) Filed: Apr. 30, 2019

(51) Int. Cl. A61K 8/81; A61K 8/49; A61Q 1/02

(52) U.S. Cl. CPC A61K 8/8152; A61Q 1/02; A61K 8/498

(57) ABSTRACT

A bodypainting ink includes a coloring agent, a solvent, and a resin. The coloring agent includes an anion coloring agent, the resin includes a cation monomer, and a molar ratio of cations, constituting the cation monomer, to anions, constituting the anion coloring agent, is 1 or more.

### FIELD

The embodiments discussed herein relate to bodypainting inks, bodypainting instruments including the bodypainting inks, and methods for manufacturing bodypainting inks.

### BACKGROUND

Compositions including an acrylic acid or a methacrylic acid derived polymeric or copolymeric component in combination with at least one water soluble organic pigment are disclosed in U.S. Patent Application Publication No. 2001-55600A1 (hereinafter, referred as Patent Document 1 (e.g., in paragraph 0007 in Patent Document 1)). Patent Document 1 discloses that long-wearing cosmetic compositions which are useful as eyeliners or, other eye or skin products can be provided in accordance with the composition disclosed in Patent Document 1.

However, bodypainting inks that are more resistant to coming off the skin and bodypainting instruments including such bodypainting inks have been conventionally demanded.

### SUMMARY

In one exemplary aspect of the present invention, bodypainting inks according to exemplary embodiments of the invention may each include a coloring agent, a solvent, and a resin, wherein the coloring agent is an anion coloring agent, the resin is constituted of a cation monomer, and the molar ratio of cations, constituting the cation monomer, to anions, constituting the anion coloring agent, is 1 or more.

In another exemplary aspect of the present invention, bodypainting instruments according to other embodiments of the invention may include the bodypainting inks according to the above-mentioned exemplary embodiments.

In other exemplary aspects of the invention, methods for manufacturing bodypainting inks according to any other exemplary embodiments of the invention may each include separating the anion coloring agent from the bodypainting ink according to the above-mentioned embodiments to thereby quantitatively measure an isolated anion coloring agent and to evaluate the resistance to coming off the skin.

In the above-mentioned exemplary embodiments, bodypainting inks that are more resistant to coming off the skin than conventional ones, as well as bodypainting instruments including such bodypainting inks, and methods for manufacturing the bodypainting inks can be provided.

The present invention will become more fully understood from the detailed description given hereinbelow. The other applicable fields will become apparent with reference to the detailed description given hereinbelow. However, the detailed description and the specific exemplary embodiment are illustrated of desired embodiments of the present invention and are described only for the purpose of explanation. Various changes and modifications will be apparent to those ordinary skilled in the art on the basis of the detailed description. The applicant has no intention to give to public any disclosed embodiments. Among the disclosed changes and modifications, those which may not literally fall within the scope of the present claims constitute, therefore, a part of the present invention in the sense of doctrine of equivalents.

### DESCRIPTION OF EXEMPLARY EMBODIMENTS

A bodypainting ink according to the present exemplary embodiment is used as a decorative (e.g., cosmetic) ink. For example, the bodypainting ink according to the present exemplary embodiment can be removed by washing away with a detergent or the like, and thus, may be used for drawing, on the skin, aesthetic decoration figures or characters, such as temporary tattoo arts. The figures or characters drawn on the skin by using the bodypainting ink of the present exemplary embodiment are preferably resistant to coming off (e.g., disappearing from) the skin for, for example, one day or longer, more preferably three days or longer, and further even more preferably about one week. The bodypainting ink according to the present exemplary embodiment does not come off the skin for about one week in a state where the sharp contours of drawn lines are maintained, whereas the bodypainting ink can be removed off the skin by washing with a detergent or the like when a user desires. According to the present exemplary embodiment, a significantly excellent bodypainting ink, which prevents a color migration (e.g., staining) onto a shirt or the like of a user until the user washes the body painting ink with a detergent or the like, can be provided.

In one other exemplary embodiment in which a bodypainting ink is prepared only from ingredients with no irritating characteristics to the eye, the bodypainting ink may be used for drawing eye shadows or eyebrows. In the other exemplary embodiment, the bodypainting ink may be used for drawing nail arts. In other exemplary embodiments, the bodypainting ink may be used for drawing marks for medical surgeries. The bodypainting inks that are more resistant to coming off the skin than conventional ones may be suitably used in any of the embodiments described above.

The bodypainting ink according to the present exemplary embodiment contains a coloring agent, a solvent, and a resin. The bodypainting ink according to the present exemplary embodiment includes an anion coloring agent as the coloring agent. An anion coloring agent is easily soluble in water, and harmfulness thereof to the human body is relatively low. Thus, an anion coloring agent may be used preferably (e.g., as a coloring agent for foods, for decoration (e.g., cosmetic use), and for medical use). As the anion coloring agent, any one of known anion coloring agents may be used arbitrarily, and the anion coloring agent may be an anion dye or an anion pigment. In the present exemplary embodiment, phloxine (i.e., red No. 104) is used as the anion coloring agent. In another exemplary embodiment, any of other anion coloring agents may be used arbitrarily, and any anion coloring agent with any color, obtained by combining a plurality of anion coloring agents may be used arbitrarily.

The bodypainting ink according to the present exemplary embodiment contains a resin constituted of a cation monomer. Here, the “resin constituted of a cation monomer” is to be understood to mean a resin constituted by polymerizing cation monomers, or a resin constituted by co-polymerizing a cation monomer and another monomer. With this configuration, an anion constituting an anion coloring agent that is easily soluble to water can be firmly bonded to a cation of the cation monomer that constitutes the resin. In addition, the resin constituted of the cation monomer firmly adheres to the skin and thus a bodypainting ink in which a coloring agent hardly comes off the skin can be obtained.

In the bodypainting ink according to the present exemplary embodiment, the molar ratio of cations, constituting the cation monomer, to anions, constituting the anion coloring agent, is configured to be 1 or more. With this configuration, substantially all of the anion coloring agent included in the bodypainting ink can be firmly bonded to cations of the cation monomers. Here, the “substantially all” is to be understood to mean an amount that is 80 wt % or more of the weight of the anion coloring agent in the bodypainting ink, for example. A bodypainting ink with such a configuration is more resistant to coming off the skin than conventional ones.

Further, in the bodypainting ink according to the present exemplary embodiment, the molar ratio of cations constituting the cation monomer to anions constituting the anion coloring agent is configured to be 2 or more. With this configuration, 90 wt % or more of the weight of the anion coloring agent included in the bodypainting ink can be firmly bonded to the cation monomers. A bodypainting ink with such a configuration is further more resistant to coming off the skin than conventional ones.

Furthermore, in the bodypainting ink according to the present exemplary embodiment, the molar ratio of cations, constituting the cation monomer, to anions, constituting the anion coloring agent, is configured to be 3 or more. With this configuration, 95 wt % or more of the weight of the anion coloring agent included in the bodypainting ink can be firmly bonded to the cation monomers. A bodypainting ink with such a configuration is further more resistant to coming off the skin than conventional ones.

The bodypainting ink according to the present exemplary embodiment may be obtained by a manufacturing method including fully mixing a resin constituted of a cation monomer and an anion coloring agent. The inventor of the present application has found that the bodypainting ink hardly comes off the skin (e.g., with significantly high resistance) when the amount of the anion coloring agent that is not bound to any cation monomers (i.e., cations constituting the cation monomers) and is isolated, is 10 wt % or less of the weight (total weight) of the bodypainting ink.

The amount of the anion coloring agent that is not bound to any cation monomers and is isolated, is more preferably 5 wt % or less of the weight (total weight) of the bodypainting ink. The amount of the anion coloring agent that is not bound to any cation monomers and is isolated is most preferably 1 wt % or less of the weight (total weight) of the bodypainting ink. Furthermore, when the amount of the anion coloring agent that is not bound to any cation monomers and is isolated is substantially 0 wt % of the weight (total weight) of the bodypainting ink, a bodypainting ink which hardly comes off the skin (e.g., with a significantly high resistance) can be obtained. Here, substantially 0 wt % is to be understood to mean an amount of, for example, less than 1 wt % of the total weight and can be deemed to be 0 wt % of the total weight.

In the present exemplary embodiment, an amine is used to obtain a cation constituting the cation monomer. In the present exemplary embodiment, the resin is constituted of an amine. With this configuration, a cation monomer can be manufactured efficiently. In addition, in the present exemplary embodiment, a methacrylate monomer is used to obtain the cation monomer. With this configuration, an amine can be introduced easily. Specifically, 2-(dimethylamino)ethyl methacrylate is used. With this exemplary configuration, a cation monomer can be manufactured efficiently.

In another exemplary embodiment, any known cation monomer may be used arbitrarily. For example, a cation monomer may include a plurality of amines to obtain the above-mentioned cations. In this case, an occurrence of the isolated anion coloring agent can be more preferably suppressed and a bodypainting ink that exhibits a significant effect as mentioned above (i.e., an effect on the resistance to coming off the skin) can preferably be obtained.

In the other exemplary embodiment, a resin constituted of an arbitrary cation monomer selected from a group consisting of acrylic resins, acrylic copolymers, and methacrylic copolymers may be used in a similar manner to the above-mentioned resin. Alternatively, a resin obtained by combining or mixing a plurality of these resins may be used in a similar manner to the above-mentioned resin. Here, the “acrylic resin” refers to a general term of resins obtainable by polymerizing an acrylic acid or a derivative thereof, and examples of the “acrylic resin” include polymers or copolymers of acrylic acid, acrylates, acrylamide, acrylonitrile, methacrylic acid, methacrylates, etc. Indeed, the “acrylic copolymer” may include polymers formed by polymerizing a plurality of different copolymers derived from acrylic monomers. When an acrylic resin is used, water-resistance of a bodypainting ink can be enhanced. In other embodiments, the cation monomer may be one constituting a styrene resin, polyvinylpyrrolidone, or the like in a similar manner to the above-mentioned acrylic resin.

The solvent contained in the bodypainting ink according to the present exemplary embodiment includes water and an alcohol. With this constitution, inhibition, by water, of bonding between an anion coloring agent that is easily soluble to water and the above-mentioned cation monomer can be more effectively suppressed in comparison with a case where the solvent is constituted only of water. In addition, in the bodypainting ink according to the present exemplary embodiment, the amount of an alcohol contained in the bodypainting ink is larger than the amount of water in the bodypainting ink. In this case, inhibition, by water, of bonding between the anion coloring agent and the above-mentioned cation monomer can be even more effectively suppressed. Therefore a more preferable bodypainting ink can be obtained. Specifically, the bodypainting ink according to the present exemplary embodiment contains water in an amount of 10 wt % of the total weight of the bodypainting ink and contains an alcohol in an amount of 40 wt % of the total weight of the bodypainting ink. With this configuration of the ratio in content of water and an alcohol, an excellent bodypainting ink that is easily applicable and has a high water-resistance can be obtained. As such, in exemplary configurations, the bodypainting ink contains water in an amount of 10 wt % or more of the total weight of the bodypainting ink and contains an alcohol in an amount of 40 wt % or more of the total weight of the bodypainting ink.

In the present exemplary embodiment, an alcohol containing 3 or less carbon atoms is used as the above-mentioned alcohol to be contained in the solvent. With this configuration, a preferable bodypainting ink with reduced irritation to human bodies can be obtained. In the present exemplary embodiment, 1-propanol is used as the above-mentioned alcohol to be contained in the solvent. In this case, a preferable bodypainting ink can be easily obtained.

In the bodypainting ink according to the present exemplary embodiment configured as described above, preferable bonding between the anion coloring agent and the above-mentioned cation monomer may be facilitated by stirring the bodypainting ink. In the present exemplary embodiment, the amount (i.e., containing weight to the total weight) of the above-mentioned isolated anion coloring agent included in the stirred bodypainting ink is quantitatively measured in separating the isolated anion coloring agent from the bodypainting ink using thin-layer chromatography, to evaluate the quality of the bodypainting ink. Since the isolated anion coloring agent is especially markedly separated from the bodypainting ink by thin-layer chromatography, the amount of the isolated anion coloring agent can be quantitatively measured. When the bodypainting ink is evaluated in such a manner, the degree of resistance to coming off the skin (i.e., quality) of the bodypainting ink can be quantitatively evaluated and the bodypainting ink can be manufactured preferably. Stirring may be continued while performing the above-mentioned evaluation until the amount of the isolated anion coloring agent reaches a predetermined amount that achieves desired qualities of the bodypainting ink. The above-mentioned stirring may be performed using any known stirring method.

In one other exemplary embodiment, the degree of resistance to coming off the skin (i.e., quality) of the stirred bodypainting ink may be evaluated more simply by similarly quantitatively measuring the amount of the isolated anion coloring agent by paper chromatography in which water is used as a developer. In the other exemplary embodiment, the degree of resistance to coming off the skin (i.e., quality) of the stirred bodypainting ink may be evaluated by similarly quantitatively measuring the amount of the isolated anion coloring agent by high performance liquid chromatography (HPLC). In this case, since the amount of the isolated anion coloring agent can be measured more precisely, a bodypainting ink that hardly comes off the skin can be preferably manufactured with accurately evaluating the quality of the bodypainting ink.

The bodypainting ink configured as described above may be used in any other embodiments by filling a bodypainting instrument that includes a tubular shaft for housing the bodypainting ink therein, an applicator disposed on the tip of the tubular shaft, and a cap for covering the applicator. With this configuration, the bodypainting ink becomes more convenient to carry around and can be more easily applied to the skin by the bodypainting instrument filled with the bodypainting ink. In addition, a configuration of the bodypainting instrument may have any configuration of known bodypainting instruments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Exemplary embodiments of this invention are described herein, including the best mode known to the inventors for carrying out the invention. Variations of those exemplary embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The exemplary invention is not limited to the exemplary embodiments detailed above. The specific configuration of each portion can be modified within the range not departing from the purpose of the exemplary invention.

The descriptions of the various exemplary embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Further, Applicant's intent is to encompass the equivalents of all claim elements, and no amendment to any claim of the present application should be construed as a disclaimer of any interest in or right to an equivalent of any element or feature of the amended claim.

1. A bodypainting ink, including:
a coloring agent;
a solvent; and
a resin,
wherein the coloring agent comprises an anion coloring agent, the resin includes a cation monomer, and a molar ratio of cations, constituting the cation monomer, to anions, constituting the anion coloring agent, is 1 or more, and
wherein the solvent includes water and an alcohol, and the coloring agent and the resin are solved in the solvent as a liquid.

2. The bodypainting ink according to claim 1, wherein the molar ratio of cations, constituting the cation monomer, to anions, constituting the anion coloring agent, is 2 or more.

3. The bodypainting ink according to claim 1, wherein the molar ratio of cations, constituting the cation monomer, to anions, constituting the anion coloring agent, is 3 or more.

4. The bodypainting ink according to claim 1, wherein an amount of the anion. coloring agent that is not bound to any cation monomers of the resin and is isolated, is 10 wt % or less of a total weight of the bodypainting ink.

5. The bodypainting ink according to claim 4, wherein the amount of the anion coloring agent that is not bound to any cation monomers of the resin and is isolated, is 5 wt % or less of the total weight of the bodypainting ink.

6. The bodypainting ink according to claim 4, wherein the amount of the anion coloring agent that is not bound to any cation monomers of the resin and is isolated, is 1 wt % or less of the total weight of the bodypainting ink.

7. The bodypainting ink according to claim 4, wherein the amount of the anion coloring agent that is not bound to any cation monomers of the resin and is isolated, is substantially 0 wt % of the total weight of the bodypainting ink.

8. The bodypainting ink according to claim 1, wherein the resin includes an amine to obtain the cations constituting the cation monomer.

9. The bodypainting ink according to claim 1, wherein the resin includes a methacrylate monomer to obtain the cation monomer.

10. The bodypainting ink according to claim 1, wherein the resin includes 2-(dimethylamino)ethyl methacrylate to obtain the cation monomer.

11. The bodypainting ink according to claim 1, wherein the resin is at least one resin selected from a group consisting of an acrylic resin, an acrylic copolymer, a methacrylic resin, and a methacrylic copolymer.

12. (canceled)

13. The bodypainting ink according to claim 1, wherein the alcohol includes an alcohol having 3 or less carbon atoms.

14. The bodypainting ink according to claim 1, wherein an amount of the alcohol contained in the bodypainting ink is larger than an amount of water contained in the bodypainting ink.

15. The bodypainting ink according to claim 1, wherein the bodypainting ink contains 10 wt % or more of water and 40 wt % or more of the alcohol.

16. The bodypainting ink according to claim 1, wherein the alcohol includes 1-propanol.

17. A bodypainting instrument including the bodypainting ink according to claim 1.

18. A method for manufacturing the bodypainting ink according to claim 1, the method including separating the anion coloring agent from the bodypainting ink to quantitatively measure an isolated anion coloring agent and to evaluate a resistance to coming off the skin.

19. The bodypainting ink according to claim 1, wherein the anion coloring agent comprises phloxine.

20. The bodypainting ink according to claim 1, wherein the resin including the cation monomer comprises one of:
a resin constituted by polymerizing cation monomers; and
a resin constituted by co-polymerizing the cation monomer and another monomer

21. The bodypainting ink according to claim 1, wherein the anion coloring agent comprises phloxine being solved in the solvent as the liquid, and
wherein the cation monomer of the resin includes 2-(dimethylamino)ethyl methacrylate being solved in the solvent as the liquid.
